(19) United States
(12) Patent Application Publication
(10) Pub. No.: US 2015/0218562 A1
(43) Pub. Date: Aug. 6, 2015

(54) WOUND TREATMENT

(71) Applicant: CoDa Therapeutics, Inc., San Diego, CA (US)

(72) Inventors: Peter Cormie, London (GB); David Becker, Hemel Hempsted (GB); David Whitmore, London (GB)

(73) Assignee: CoDa Therapeutics, Inc., San Diego, CA (US)

(21) Appl. No.: 14/396,722

(22) PCT Filed: Apr. 23, 2013
(86) PCT No.: PCT/GB2013/000178

§ 371 Date: Oct. 23, 2014

(30) Foreign Application Priority Data
Apr. 23, 2012 (GB) 1207056.1

(51) Int. Cl. C12N 15/113

(52) U.S. Cl. CPC C12N 15/1137; C12N 15/113; C12N 2310/11

(57) ABSTRACT

The present invention concerns an isolated polynucleotide comprising a nucleotide sequence having substantial homology to any of the following nucleotide sequences: catcgttatgggacta (SEQ ID NO: 2), cattcttgatccttcc (SEQ ID NO: 1), cttttcaatctgactg SEQ ID NO: atgaaaatactcataa (SEQ ID NO: 5), gtgataaaagaaccat (SEQ ID NO: 10), gggttcatgaaagtga (SEQ ID NO: 11), gatgaccctcttatcc (SEQ ID NO: 8), tggaaggaatgtctgg (SEQ ID NO: 4), gcatctgcttccaaca (SEQ ID NO: 3), catcgttaggctagctacaacgatgggacta (SEQ ID NO: 9), tccaccaaggctagctacaacgaccatcaaa (SEQ ID NO: 12), gtcaacaaggctagctacaacgatgagctca (SEQ ID NO: 13), and cttttcaaggctagctacaacgactgactgt (SEQ ID NO: 6), and their use in the treatment of wounds.

### CROSS-REFERENCE TO RELATED APPLICATION

This application is a 371 National Stage Application of International Application No. PCT/GB2013/000178, filed on Apr. 23, 2013, which claims priority to Great Britain Patent Application No. 1207056.1, which was filed on Apr. 23, 2012, the contents of which are each incorporated herein by reference in their entireties.

### FIELD OF THE INVENTION

The invention relates to compositions for the treatment of wounds, especially chronic wounds.

### BACKGROUND TO THE INVENTION

Most skin wounds heal naturally over the course of a few days without any problems. However, in the elderly and in diabetics wounds do not heal well and are prone to infections and often end up as chronic non-healing ulcers. These wounds have a very negative impact on the quality of life and morbidity of the sufferers. There are currently no effective therapeutic treatments for these chronic wounds, which cost health care authorities billions to treat each year. This is a very serious problem and one that is set to get worse with the growing numbers of elderly and diabetics in our population.

Understanding the factors that control the normal healing process and finding ways to improve it is likely to help us to drive the healing in these chronic wound conditions. One of the key factors in the failure of chronic wounds to heal is the migration of fibroblasts from surrounding tissues into the wound bed to form granulation tissue.

The inventors have recently found that the circadian clock plays an important role in wound healing. This is novel, unexpected and could lead to a new therapeutic approach to wound healing.

### SUMMARY OF THE INVENTION

The invention provides an isolated polynucleotide capable of hybridising to CLOCK or BMAL mRNA. In particular, the invention provides an isolated polynucleotide capable of hybridising with CLOCK or BMAL mRNA and suppressing the activity of that mRNA. The polynucleotide is preferably antisense to CLOCK or BMAL mRNA. Preferably the polynucleotide comprises a nucleotide sequence having substantial homology to any of the following nucleotide sequences:

catcgttatgggacta, cattcttgatccttcc, cttttcaatctgac
tg, atgaaaatactcataa, gtgataaaagaaccat,
gggttcatgaaagtga, gatgaccctcttatcc, tggaaggaatgtct
gg, gcatctgcttccaaca, catcgttaggctagctacaacgatggga
cta, tccaccaaggctagctacaacgaccatcaaa, gtcaacaaggct
agctacaacgatgagctca,
and
cttttcaaggctagctacaacgactgactgt tccaccaaccatcaaa,
gtcaacaatgagctca,
and
cttttcaactgactgt.

The term polynucleotide is also considered, herein, to encompass any molecule which has a base sequence with a structure similar to that of DNA or RNA so that the base sequence of the molecule can base pair with a complementary base sequence. Polynucleotides may include, but are not limited to oligodeoxyribonucleotides or oligoribonucleotides, phosphorodiamidate morpholino oligonucleotides (PMO), 2′-O-methyl (2′OMe) oligonucleotides, locked nucleic acids (LNA) or peptide nucleic acids (PNA), oligonucleotides containing phosporothioate bonds, 2′-fluoro oligonucleotides, hexitol nucleic acid, 2′-O-methoxyethyl oligonucleotide, 2′-O-allyl oligonucleotide, 2′-O-propyl oligonucleotide, 2′-O-pentyl oligonucleotide, or oligonucleotides with multiple modifications, such as those comprising phosphorothioate bonds and fluoro or allyl groups. The polynucleotide may, for example be RNA, including miRNA, shRNA and siRNA as well as precursors that can be processed to produce such molecules such as a pri-miRNA or pre-siRNA. Alternatively, it may be single stranded DNA.

The polynucleotide of the invention can be used to bind to CLOCK or BMAL mRNA and to suppress its activity. The sequences of CLOCK and BMAL mRNA are provided in FIGS. 5 and 6. The polynucleotide therefore preferably comprises a nucleotide sequence that is complementary to the sequence of a region of the CLOCK or BMAL genes. The term “complementary” means that the majority of the bases in a first sequence are complementary to a second sequence. However, absolute complementarity is not required, it is sufficient for the polynucleotide to be able to form a stable duplex with CLOCK or BMAL mRNA at physiological temperatures. For example, the two sequences will still be able to base pair if there are a small number of mismatched bases or a small “bulge” of non-paired bases in the first sequence. For example, if there are five or fewer mismatched bases or a bulge of five or fewer bases, the two base sequences should still be able to base pair. Preferably, there is no “bulge” of non-paired bases. Preferably, there are four or fewer mismatched bases, more preferably, three or fewer mismatched bases, even more preferably, two or fewer mismatched bases, more preferably still, one or fewer mismatched bases and, most preferably, no mismatched bases.

The polynucleotide of the invention preferably has or comprises a nucleotide sequence having substantial homology to one of catcgttatgggacta (SEQ ID NO. 2; also referred to herein as 180), cattcttgatccttcc (SEQ ID NO. 1; also referred to herein as 722), cttttcaatctgactg (SEQ ID NO. 7; also referred to herein as 750), atgaaaatactcataa (SEQ ID NO. 5; also referred to herein as 1639), gtgataaaagaaccat (SEQ ID NO. 10; also referred to herein as 1749), gggttcatgaaagtga (SEQ ID NO. 11; also referred to herein as 1782), gatgaccctcttatcc (SEQ ID NO. 8; also referred to herein as 2044), tggaaggaatgtctgg (SEQ ID NO. 4; also referred to herein as 2056), gcatctgcttccaaca (SEQ ID NO. 3; also referred to herein as 2337), catcgttaggctagctacaacgatgggacta (SEQ ID NO. 9; also referred to herein as 23), tccaccaaggctagctacaacgaccatcaaa (SEQ ID NO. 12; also referred to herein as 15), gtcaacaaggctagctacaacgatgagctca (SEQ ID NO. 13; also referred to herein as 8), and cttttcaaggctagctacaacgactgactgt (SEQ ID NO. 6; derived from 180). The term “substantial homology” preferably means at least 85%, 87%, 90%, 92% or 95% homology. The polynucleotide preferably has or comprises a sequence differing by no more than 1, 2, 3, 4 or 5 bases.

In particular, the polynucleotide is preferably antisense to at least part of CLOCK or BMAL mRNA. It can preferably hybridise to CLOCK or BMAL mRNA and inhibit the expression of CLOCK or BMAL Inhibition of CLOCK or BMAL may be brought about by interfering with or altering one of the steps of expression, such as transcription, processing, transportation, translation or degradation of mRNA. In particular, the polynucleotides of the invention may bind to CLOCK or BMAL mRNA and physically prevent it from being translated. Alternatively, they may bind to the mRNA and cause it to be cut or otherwise broken down. The polynucleotide may hybridise to an entire mRNA or, more preferably to part of it.

The polynucleotide is preferably at least 5, 6, 7, 8, 9, 10, 11 or 12 bases in length. It may be up to around 40, 60, 80, 100, 150 or 200 bases in length.

In one embodiment, the polynucleotide is preferably between 14 and 34 bases in length and more preferably between 15 and 32 bases in length. It is particularly preferred that the polynucleotide is between 15 and 20, 19, 18 or 17 bases in length. It is most preferably 16 bases in length. Alternatively, the polynucleotide is preferably between 28, 29, 30 or 31 and 31, 32, 33, or 34 bases in length. It is most preferably 30, 31 or 32 bases in length, especially 31 bases.

Alternatively, in another embodiment, the polynucleotide may comprise a binding region of nucleotides flanked on one or both ends by a flanking region. The binding region comprises a sequence of nucleotides which hybridises to CLOCK or BMAL, especially to at least part of CLOCK or BMAL mRNA. The binding region may have or comprise a sequence having substantial homology to part of one of catcgttatgggacta, cattcttgatccttcc, cttttcaatctgactg, atgaaaatactcataa, gtgataaaagaaccat, gggttcatgaaagtga, gatgaccctcttatcc, tggaaggaatgtctgg, gcatctgcttccaaca, tccaccaaccatcaaa, gtcaacaatgagctca, cttttcaactgactgt, catcgttaggctagctacaacgatgggacta, tccaccaaggctagctacaacgaccatcaaa, gtcaacaaggctagctacaacgatgagctca, and cttttcaaggctagctacaacgactgactgt. It is preferably between 14 and 34 bases in length and more preferably between 15 and 32 bases in length. It is particularly preferred that the binding region is between 15 and 20, 19, 18 or 17 bases in length. It is most preferably 16 bases in length. Alternatively, the binding region is preferably between 28, 29, 30 or 31 and 31, 32, 33, or 34 bases in length. It is most preferably 30, 31 or 32 bases in length, especially 31 bases. The flanking region is preferably between 10, 15, 20 and 25 bases and 20, 25, 30, 35, 40 and 45 bases in length. The flanking region may or may not be able to hybridise CLOCK or BMAL mRNA.

In another embodiment, the polynucleotide may comprise two binding regions of nucleotides, particularly of between 6, 7, 8, 9 and 10 bases and 20, 19, 18, 17 and 16 bases in length, the binding regions flanking a catalytic region of bases, particularly of between 8, 9, 10, 11 and 12 bases in length. The binding regions are able to hybridise with CLOCK or BMAL, especially CLOCK or BMAL mRNA. It is particularly preferred that the binding regions hybridise to contiguous or very close regions of CLOCK or BMAL mRNA. The binding regions may each preferably comprise at least 8 contiguous nucleotides from the following sequences: catcgttatgggacta, cattcttgatccttcc, cttttcaatctgactg, atgaaaatactcataa, gtgataaaagaaccat, gggttcatgaaagtga, gatgaccctcttatcc, tggaaggaatgtctgg, gcatctgcttccaaca, tccaccaaccatcaaa, gtcaacaatgagctca, cttttcaactgactgt. The two binding regions found in one polynucleotide preferably both comprise a nucleotide sequence taken from one of: catcgttatgggacta, cattcttgatccttcc, cttttcaatctgactg, atgaaaatactcataa, gtgataaaagaaccat, gggttcatgaaagtga, gatgaccctcttatcc, tggaaggaatgtctgg, gcatctgcttccaaca, tccaccaaccatcaaa, gtcaacaatgagctca, cttttcaactgactgt, especially so that if the binding regions are placed contiguously they form one of those sequences.

The catalytic region comprises a sequence of nucleotides having catalytic, especially ligating activity. In one embodiment, the catalytic region comprises the following nucleotide sequence:

ggctagctacaacga.

Accordingly, the polynucleotide of the invention preferably comprises or consists of one or more of the following sequences:

catcgttatgggacta,
cattcttgatccttcc,
cttttcaatctgactg,
atgaaaatactcataa,
gtgataaaagaaccat,
gggttcatgaaagtga,
gatgaccctcttatcc,
tggaaggaatgtctgg,
gcatctgcttccaaca,
catcgttaggctagctacaacgatgggacta,
tccaccaaggctagctacaacgaccatcaaa,
gtcaacaaggctagctacaacgatgagctca,
and
cttttcaaggctagctacaacgactgactgt.

In a further embodiment, the polynucleotide of the invention preferably comprises or consists of one or more of the following sequences:

(SEQ ID NO. 14; derived from 722)
ccttggtgttctgcatattctaaccttcca,
(SEQ ID NO. 15; derived from 722)
tccttccttggtgttctgcatattctaacc,
(SEQ ID NO. 16; derived from 722)
atccttccttggtgttctgcatattctaac,
(SEQ ID NO. 17; derived from 722)
gatccttccttggtgttctgcatattctaa,
(SEQ ID NO. 18; derived from 722)
ttccttggtgttctgcatattctaaccttc,
(SEQ ID NO. 19; derived from 722)
tccttggtgttctgcatattctaaccttcc,
(SEQ ID NO. 20; derived from 2337)
atctgcttccaagaggctcatgatgacagc,
(SEQ ID NO. 21; derived from 722)
cttggtgttctgcatattctaaccttcca,
(SEQ ID NO. 22; derived from 722)
ccttccttggtgttctgcatattctaacc,
(SEQ ID NO. 23; derived from 722)
cttccttggtgttctgcatattctaacc,
(SEQ ID NO. 24; derived from 2056)
gagtccctccatttagaatcttcttgcc,
(SEQ ID NO. 25; derived from 2337)
gcttccaagaggctcatgatgacagcca,
(SEQ ID NO. 26; derived from 722)
ttccttggtgttctgcatattctaacc,
(SEQ ID NO. 27; derived from 115)
tctgtaaaacttgcctgtgacattc,
(SEQ ID NO. 28; derived from 115)
gtctgtaaaacttgcctgtgacattc,
(SEQ ID NO. 29; derived from 722)
tccttggtgttctgcatattctaacc,
(SEQ ID NO. 30; derived from 180)
gttactgggactacttgatccttgg,
and
(SEQ ID NO. 31; derived from 2056)
gagtccctccatttagaatcttcttg.

The polynucleotide is indicated as containing the base thymine (T). However, as will be appreciated by one skilled in the art, T can be replaced with the base uracil (U). Whether the base T or U is selected will depend on the type of molecule containing the sequence. For example, if the molecule is a DNA molecule or a PMO, the base may be T whereas if the molecule is a RNA molecule, the base may be U. Therefore, the molecule of the invention is not limited to a sequence containing T but can also comprise a sequence containing U since the function of the base at these positions is to bind to the base A, a function which both U and T can fulfill.

Preferably, the polynucleotide is isolated so that it is substantially free from other compounds or contaminants.

The polynucleotide may be conjugated to or complexed with an entity, especially an entity which helps target the polynucleotide to the required site of action.

Also provided by the invention is a vector comprising a polynucleotide as previously described. The vector may comprise components required for expression of the polynucleotide in a mammalian cell. Any appropriate vector can be used, including, for example, an adenoviral vector, an adeno-associated viral vector, or a lentiviral vector. Also provided is a cell comprising the vector of the invention, especially a mammalian, bacterial or insect cell. The cell is preferably not a human embryonic cell, or if it is, it may preferably be produced without the destruction of a human embryo.

Further provided is a pharmaceutical composition comprising one or more of the polynucleotides or one or more of the vectors described previously and a pharmaceutically acceptable carrier or excipient. In particular, the composition may comprise a carrier which enables the polynucleotide to be delivered to the relevant site for use. The carrier may target a particular site or otherwise improve delivery to that site. When the pharmaceutical composition comprises a polynucleotide, it may also comprise an excipient which stabilises the polynucleotide. Such stabilisers are well known in the art. Any appropriate stabiliser may be used. The pharmaceutical composition may also comprise one or more other therapeutic agents, especially one or more agents effective in treating wounds.

Pharmaceutical compositions of this invention comprise any of the molecules of the present invention, and pharmaceutically acceptable salts thereof, with any pharmaceutically acceptable carrier, adjuvant or vehicle. Pharmaceutically acceptable carriers, adjuvants and vehicles that may be used in the pharmaceutical compositions include, but are not limited to, ion exchangers, alumina, aluminium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulphate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The pharmaceutical compositions of this invention may be administered orally, parenterally, by inhalation spray, topically, rectally, nasally, buccally, vaginally or via an implanted reservoir. Preferably, the pharmaceutical compositions are administered orally or by injection. The pharmaceutical compositions may contain any conventional non-toxic pharmaceutically-acceptable carriers, adjuvants or vehicles. The term parenteral as used herein includes subcutaneous, intracutaneous, intravenous, intramuscular, intra-articular, intrasynovial, intrasternal, intrathecal, intralesional and intracranial injection or infusion techniques. Preferably, the route of administration of the composition is transdermal or intrathecal administration.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant such as Ph. Helv or a similar alcohol.

The pharmaceutical compositions of this invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavouring and/or colouring agents may be added.

The pharmaceutical compositions of this invention may also be administered in the form of suppositories for rectal administration. These compositions can be prepared by mixing a molecule of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is especially useful when the desired treatment involves areas or organs readily accessible by topical application. For application topically to the skin, the pharmaceutical composition should be formulated with a suitable ointment containing the active components suspended or dissolved in a carrier. Carriers for topical administration of the molecules of this invention include, but are not limited to, mineral oil, liquid petroleum, white petroleum, propylene glycol, polyoxyethylene polyoxypropylene compound, poloxamers, agar, emulsifying wax and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilising or dispersing agents known in the art.

Further provided is a polynucleotide according to the invention, or a pharmaceutical composition according to the invention, for use in therapy, especially for the treatment of wounds. Also provided is the use of a polynucleotide according to the invention in the preparation of a medicament for the treatment of wounds. Further provided is a vector comprising a promotor or repressor of CLOCK or BMAL for use in the modulation of wound healing.

The term wound is intended to encompass all types of wound, but the polynucleotides and compositions of the invention are particularly useful for treating chronic or slow healing wounds. The polynucleotides and compositions of the invention may be for the treatment of any wound, whether or not it is healing at the expected or desired rate, but are particularly useful for the treatment of wounds that are healing more slowly than desired. Accordingly, the polynucleotides or compositions may be used to speed up wound healing.

The wound to be treated may be a wound caused by any of a wide range of tissue injuries, such as, but not limited to incisions, lacerations, burns, ulcers, punctures, abrasions and surgical wounds.

The polynucleotides or compositions are useful for treating wounds in any subject, especially mammals, in particular primates, domestic species and farm animals. It is especially preferred that the subject is a human. The polynucleotides or compositions are particularly useful for treating individuals who are likely to suffer delayed or slow wound healing and those at increased risk of chronic wounds. They are also especially useful for treating those more at risk of infection of a wound and those less likely to be able to recover easily from such an infection. In particular, the polynucleotides or compositions are useful for treating, for example, the elderly, the very young, immune compromised subjects, diabetic subjects and subjects who have difficulty moving.

Treating a wound preferably means improving the rate or quality of healing of a wound, such that the wound heals, for example, more quickly, less painfully, with less inflammation or with less scarring than if the polynucleotides or compositions were not used. Wound healing is likely to mean the closure of a wound, or the replacement of wound tissue with normal healthy tissue, or with scar tissue, or a combination of the two.

Also provided is an agent which alters, increases or reduces the expression or function of CLOCK or BMAL or the CLOCK or BMAL pathway for use treating wounds, or the use of such an agent in the manufacture of a medicament for treating a wound. Such agents include molecules which change the expression levels of CLOCK or BMAL themselves, or which change the expression levels of members of the CLOCK and BMAL pathways either up or downstream of CLOCK or BMAL. In particular, such agents are agents capable of changing the control of circadian clock in the cells to which they are administered, especially agents that are able to mimic the knocking down or out of CLOCK or BMAL, or mimic the effect of culturing cells in extended light. The agents may target CLOCK or BMAL, or may target other genes involved in the control of the circadian clock, such as PERIOD, CRYPTOCHROME and the REV-ERBs. Alternatively, enzymes such as the clock regulatory kinases could be targeted. Such genes may be targeted by for example the use of antisense RNAs directed at the mRNA of the genes, or using small molecules. Methods and molecules for modulating the CLOCK and BMAL pathways and the associated genes are described in the prior art (A small molecule modulates circadian rhythms through phosphorylation of the period protein. Lee J W, Hirota T, Peters E C, Garcia M, Gonzalez R, Cho C Y, Wu X, Schultz P G, Kay S A. Angew Chem Int Ed Engl. 2011 Nov. 4; 50(45):10608-11. doi: 10.1002/anie.201103915. Epub 2011 Sep 26; High-throughput chemical screen identifies a novel potent modulator of cellular circadian rhythms and reveals CKIα as a clock regulatory kinase. Hirota T, Lee J W, Lewis W G, Zhang E E, Breton G, Liu X, Garcia M, Peters E C, Etchegaray J P, Traver D, Schultz P G, Kay S A. PLoS Biol. 2010 Dec. 14; 8(12):e1000559; High-throughput screening and chemical biology: new approaches for understanding circadian clock mechanisms. Hirota T, Kay S A. Chem Biol. 2009 Sep. 25; 16(9):921-7; A chemical biology approach reveals period shortening of the mammalian circadian clock by specific inhibition of GSK-3beta. Hirota T, Lewis W G, Liu A C, Lee J W, Schultz P G, Kay S A. Proc Natl Acad Sci USA. 2008 Dec. 30; 105(52):20746-51. Epub 2008 Dec. 22; Regulation of circadian behaviour and metabolism by synthetic REV-ERB agonists. Solt L A, Wang Y, Banerjee S, Hughes T, Kojetin D J, Lundasen T, Shin Y, Liu J, Cameron M D, Noel R, Yoo S H, Takahashi J S, Butler A A, Kamenecka T M, Burris T P, Nature. 2012 Mar. 29. doi: 10.1038/nature11030; and Identification of diverse modulators of central and peripheral circadian clocks by high-throughput chemical screening. Chen Z, Yoo S H, Park Y S, Kim K H, Wei S, Buhr E, Ye Z Y, Pan H L, Takahashi J S. Proc Natl Acad Sci USA. 2012 Jan. 3; 109(1):101-6. Epub 2011 Dec. 19.) Agents that may be used include longdaysin, LH846 and lithium.

Also provided is a wound dressing comprising a polynucleotide or pharmaceutical composition according to the invention.

The wound dressing according to the invention may be any dressing suitable for application to a wound. It includes topical dressings for external wounds as well as dressings, supports and scaffolds suitable for applying to internal wounds. Such dressings are well known in the art. Appropriate wound dressings include, but are not limited to dressings comprising woven textiles or plastics, hydrogels, agars, and foams. The polynucleotides, compositions or agents of the invention may be dispersed in the wound dressing in any appropriate way, for example being dispersed in or on a top sheet of the wound dressing, or throughout the dressing. The polynucleotides, compositions or agents may be appropriately formulated to allow storage and release, being for example, freeze dried or encapsulated in a vesicle or microcapsule. The wound dressing may comprise additional materials to assist healing, for example to reduce antigenicity and immunogenicity.

A related aspect of the invention provides a method of treating wound comprising administering a therapeutically effective amount of one or more of a polynucleotide, a vector or another agent that alters the expression, or function of one or both of CLOCK or BMAL or a pharmaceutical composition as described to a subject having a wound. A therapeutically effective amount is an amount sufficient to achieve a desired effect, such the improvements in wound healing described above.

The invention will now be described in detail, by way of example only, with reference to the drawings.

### DESCRIPTION OF THE DRAWINGS

FIG. 1 shows images of full thickness 3 day wounds treated with vehicle control Pluronic gel or Pluronic gel containing a variety of 100 uM antisense sequences against Bmal. The bar graph shows the normalized areas of the wound as measured from the macroscopic images. The images below show median examples of each of the sequences. Scale bar 5 mm.

FIG. 2 shows images of full thickness 3 day wounds treated with vehicle control Pluronic gel or Pluronic gel containing a variety of 100 uM antisense sequences against 2 Clock and 1 Bmal targets. The bar graph shows the normalized areas of the wound as measured from the macroscopic images. Bmal180 wounds are significantly smaller than control. The images below show median examples of each of the sequences. Scale bar 5mm.

FIGS. 3 shows migration rates of selected CLOCK and BMAL1 shRNA-transfected NIH 3T3 fibroblasts. Images of 3T3 cell scratch wound migration assays in wild type WT cells and those transfected with shRNA against CLOCK. The white line shows the position of the leading edge of cells at the time of wounding and the black line shows the leading edge 4 hours later. The cells transfected with Clock shRNA migrate faster and have longer lamellipodia. Only 2 of the shRNA constructs (CLOCK 95684 and CLOCK 95685) were pre-validated by Sigma Aldrich. The rest were sequences predicted to be amenable to RNA interference technology. Therefore, not every shRNA construct was effective at increasing migration rate. Both of the pre-validated constructs (CLOCK 98684 and CLOCK 95685) were effective at increasing migration rate, and transfection with at least one of the previously unvalidated constructs (BMAL 95056) resulted in a similar enhancement of migration.

FIG. 4A shows representative images from a time lapse scratch wound assay performed at ZT1 hours in the circadian cycle. The white line shows the leading edge at the time of wounding and the red shows how far it has migrated in 4 hours. Graph shows the rate of migration at different times of the circadian day. Cells can be seen to migrate significantly faster at ZT13-17.

FIG. 4B shows representative images from a time lapse scratch wound assay performed on wild type cells and cells expressing dominant negative CLOCK. The white line shows the leading edge at the time of wounding and the red shows how far it has migrated in 4 hours. Cells expressing dominant negative CLOCK migrate significantly faster than control cells as shown in C—a similar effect of enhanced migration can be achieved if cells are kept in constant light LL to disrupt the clock compared to a normal light dark cycle LD.

FIG. 4D shows confocal microscope images of wild type cells and cells expressing dominant negative clock, 4 hours after a scratch wound. The red actin staining shows a belt of actin at the leading edge of wild type cells which is not seen in the CLOCK DN cells which show longer lamellipodia as the migrate forward faster.

FIG. 5 shows the nucleotide sequence of mouse BMAL mRNA (SEQ ID NO. 32; translation is SEQ ID NO. 33).

FIG. 6 shows the nucleotide sequence of human CLOCK mRNA (SEQ ID NO. 34).

FIG. 7 shows the nucleotide sequence of mouse CLOCK mRNA (SEQ ID NO. 35).

FIG. 8 shows the nucleotide sequence of Human arnt1 mRNA (SEQ ID NO. 36; translation is SEQ ID NO. 37).

FIG. 9 shows a Western blot of Bmal1 protein levels with and without treatment with antisense sequences. Column 1 concerns treatment with tccttccttggtgttctgcatattctaacc (SEQ ID NO. 15). Column 2 concerns treatment with atccttccttggtgttctgcatattctaac (SEQ ID NO. 16). Column 3 concerns treatment with gatccttccttggtgttctgcatattctaa (SEQ ID NO. 17). Column C is a control containing no nucleotide.

FIG. 10 shows a graphical representation of the Western blot of FIG. 9 following normalisation to α tubulin. α tubulin provides a standardised level of expression.

### DETAILED DESCRIPTION OF THE INVENTION

### EXAMPLES

The inventors have inhibited clock function by targeting the expression of BMAL and/or CLOCK proteins, critical components of the core circadian clock, with CLOCK and/or BMAL-specific siRNA and also antisense oligodeoxynucleotide. They have found that they are able to significantly enhance the rate of migration of fibroblasts by interfering with BMAL and/or CLOCK proteins. The model works in Zebrafish, mouse and human cell lines and mouse models in vivo. A range of accessible antisense sites for mouse and human BMAL and CLOCK have now been identified, developed and tested in cells and in animal models of wound healing as proof of principle. Further, immunostaining suggests that CLOCK and BMAL are over-expressed in the wound edge of diabetic rat wounds and human diabetic foot ulcers and venous leg ulcers.

The inventors have also shown that the speeding migration phenomenon when the CLOCK is targeted is true in mammalian cells as well as in zebrafish cells where it was first seen. It is also effective in promoting healing in mouse excissional wound healing models.

The inventors have identified accessible sites on the mRNA of mouse and human CLOCK and BMAL and designed and tested deoxyribozymes and antisenses to these sites. 8 BMAL and 3 CLOCK sequences have proved effective and are able to speed migration of mouse fibroblasts in culture and have proved effective in mouse models of wound healing.

### Example 1

### A Circadian Rhythm in Wound Healing Rate

We cultured zebrafish PAC2 cells to confluence on a light-dark (LD), induced scratch wounds, and monitored the rate of cell migration using time lapse microscopy. We observed a circadian rhythm in migration rate, with cells migrating fastest at zeitgeber time (ZT) 15, just after dusk. Migration rates were slowest at ZT3, just after dawn. It would appear, therefore, that the inherent circadian clock in each cell impacts upon their ability to migrate after scratch wounding.

### Stopping the Clock Enhances Wound Healing

Our zebrafish cell cultures provide an attractive model system with which to probe aspects of circadian clock function, and in particular the impact that clock function has on basic cellular processes. Each cell in culture contains a circadian clock that is reset each day by the LD cycle. We created a cell line that lacks a functional circadian clock by over-expressing a dominant negative form of the zebrafish CLOCK1 gene (hereafter, ΔCLK). Our interest in cell migration led us to carry out a series of experiments in which scratch wounds were induced in confluent cell monolayers and we monitored the migration response of cells at the wound edge.

After wounding, cells extend lamellipodia and migrate into the wound bed to effect healing. When we compare WT to ΔCLK migration, we see that ΔCLK cells migrate approximately twice as fast as WT cells, and extend much more extensive lamellipodia. In another series of experiments, we cultured cells in constant light (LL) conditions, a treatment known to stop the clock in zebrafish cells. As in ΔCLK cells, cells grown on LL migrate faster and extend much larger lamellipodia. The circadian clock, therefore, clearly exerts some control over the actin cytoskeleton, which is critical for migration.

### Example 2

### Stopping the Clock: NIH 3T3 Fibroblasts

NIH 3T3 fibroblasts also contain circadian clocks, though without a synchronizing stimulus these clocks are not synchronous at the population level in culture. To investigate the role of the circadian clock in NIH 3T3 migration after scratch wounding, we created stable cell lines expressing shRNA constructs against mouse CLOCK and BMAL1, and carried out scratch wound assays.

In some cases, the rate of migration in shRNA-transfected cell lines was greater than for WT cells, and we frequently saw enhanced lamellipodial extension in these cells. This data suggests that the circadian clock plays a role in control of migration in mouse NIH 3T3 cells.

### Example 3

### Design of Antisense ODNs Against Mouse and Human CLOCK and BMAL1

### 1. Deoxyribozymes.

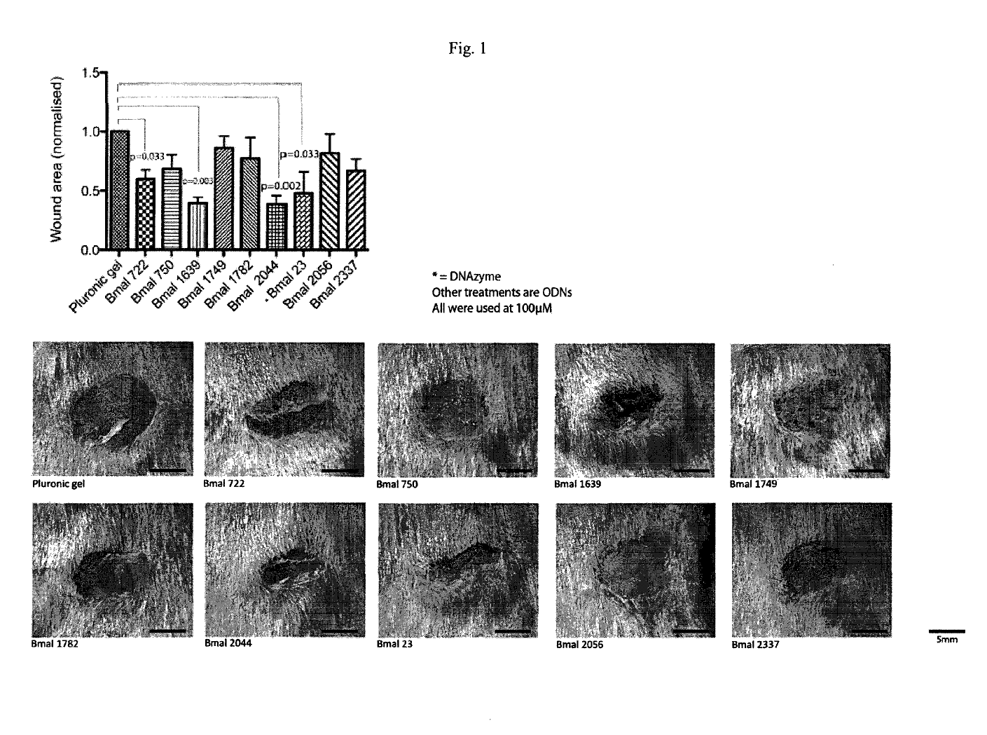

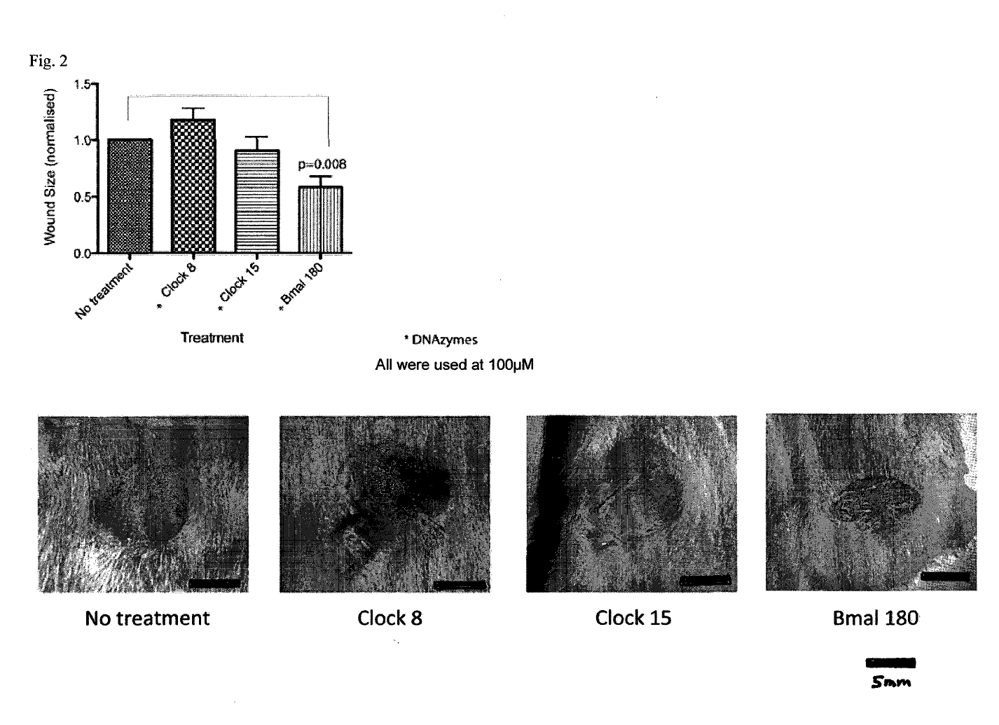

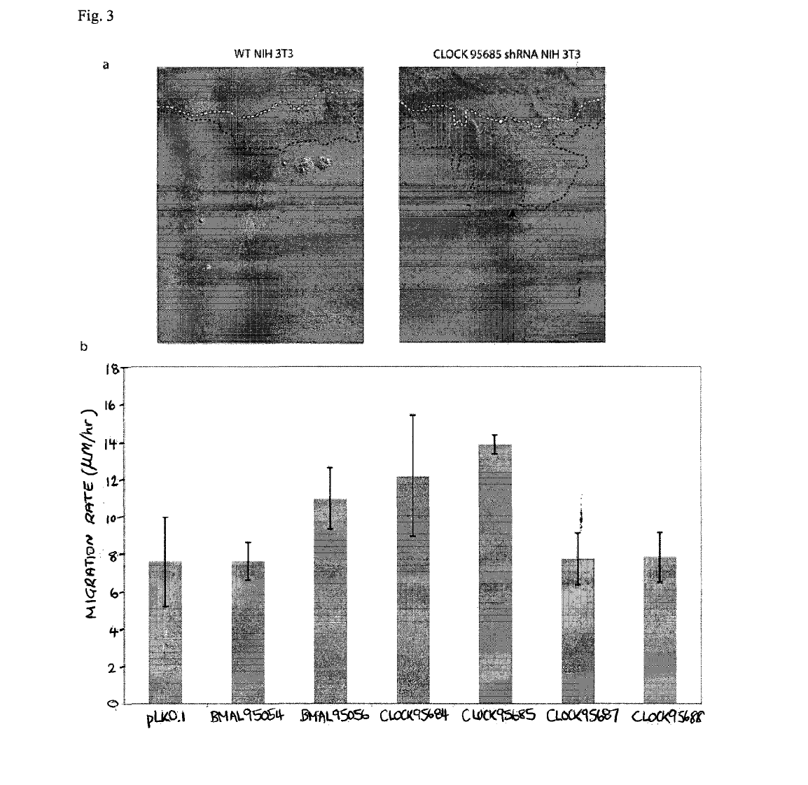

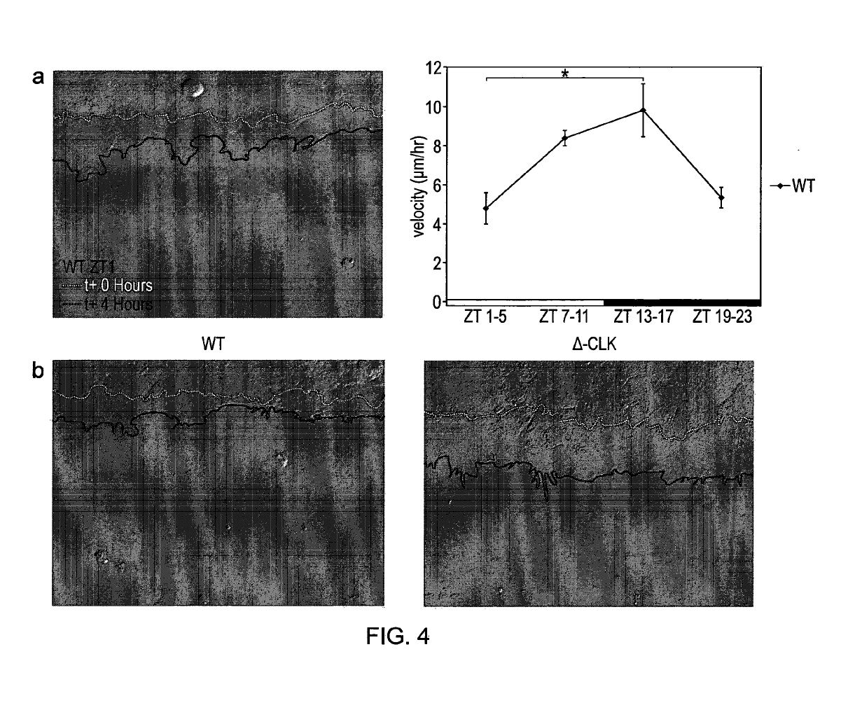

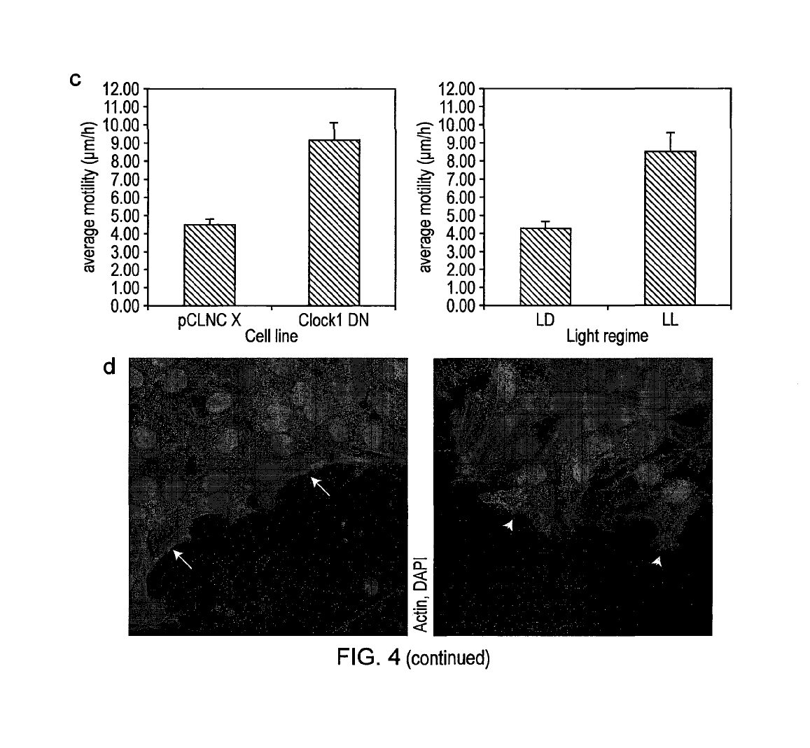

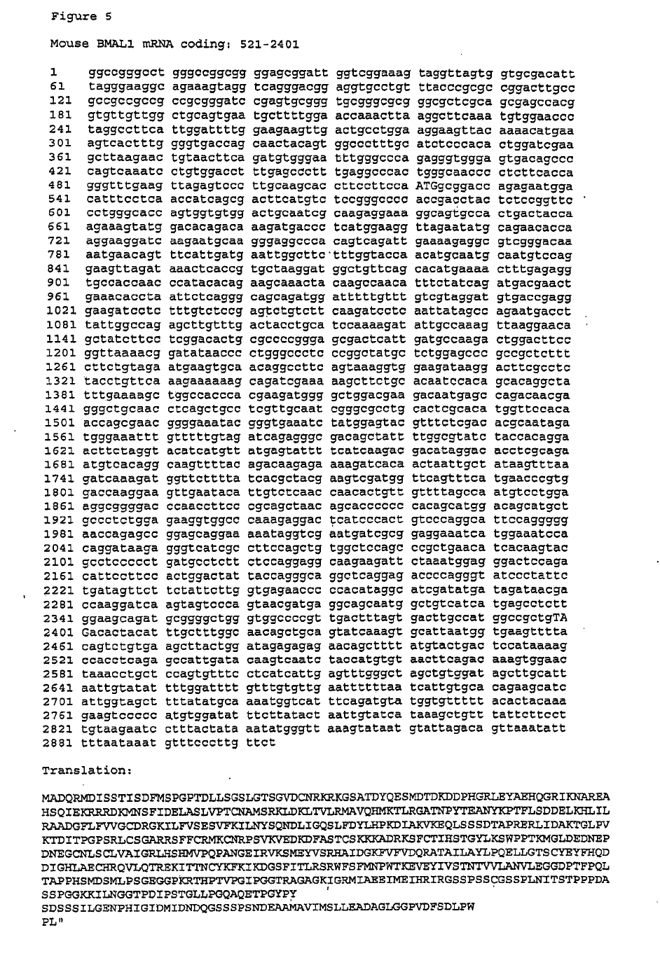

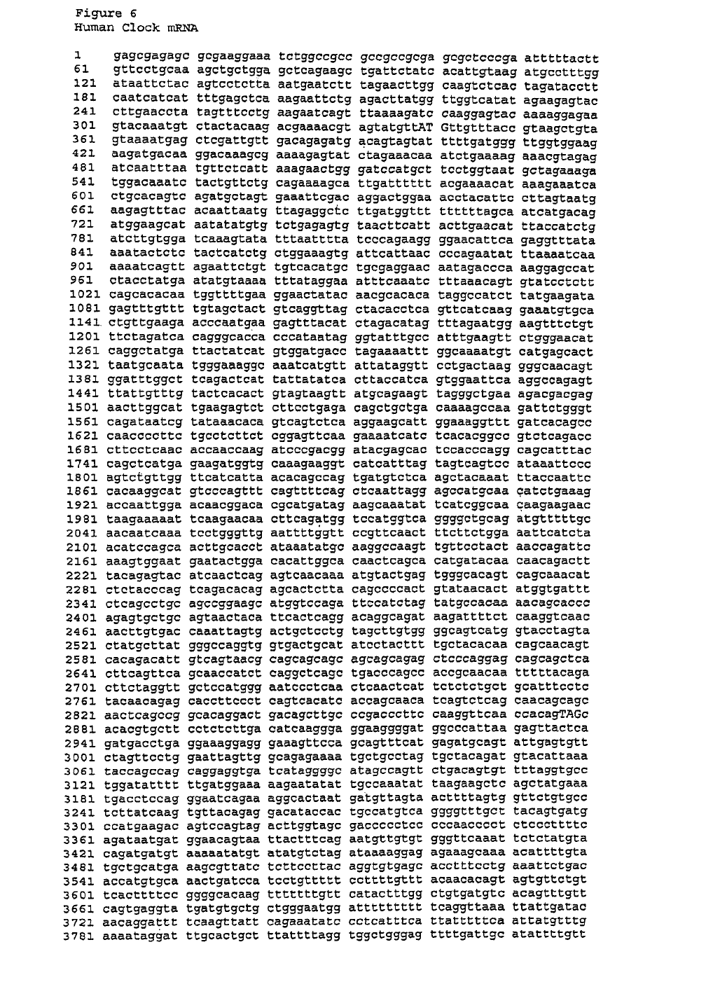

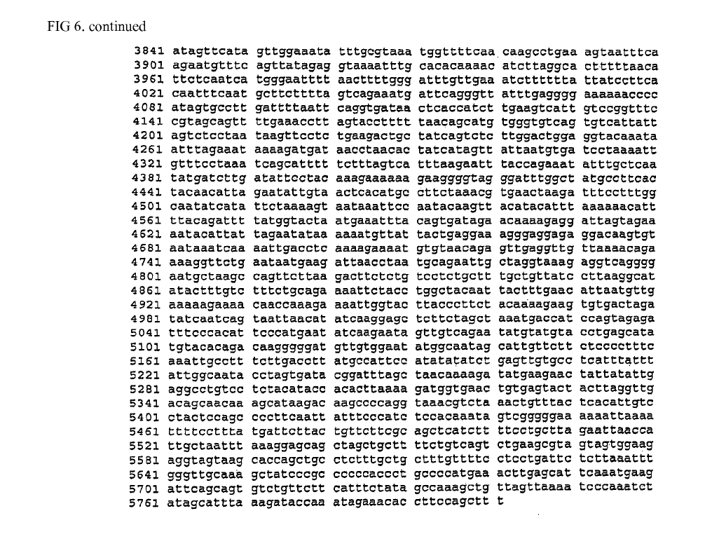

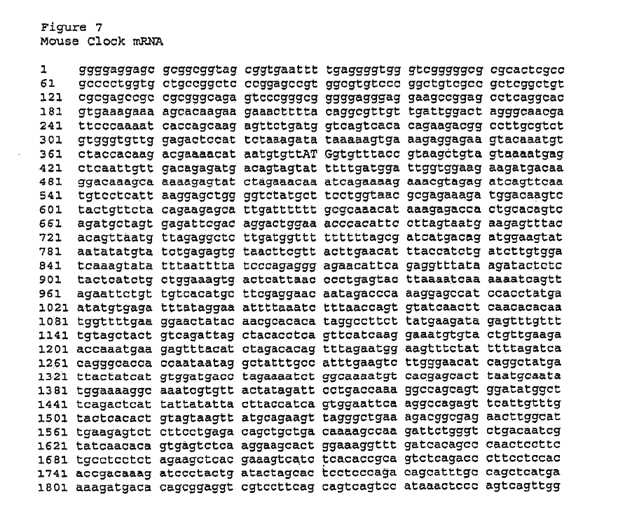

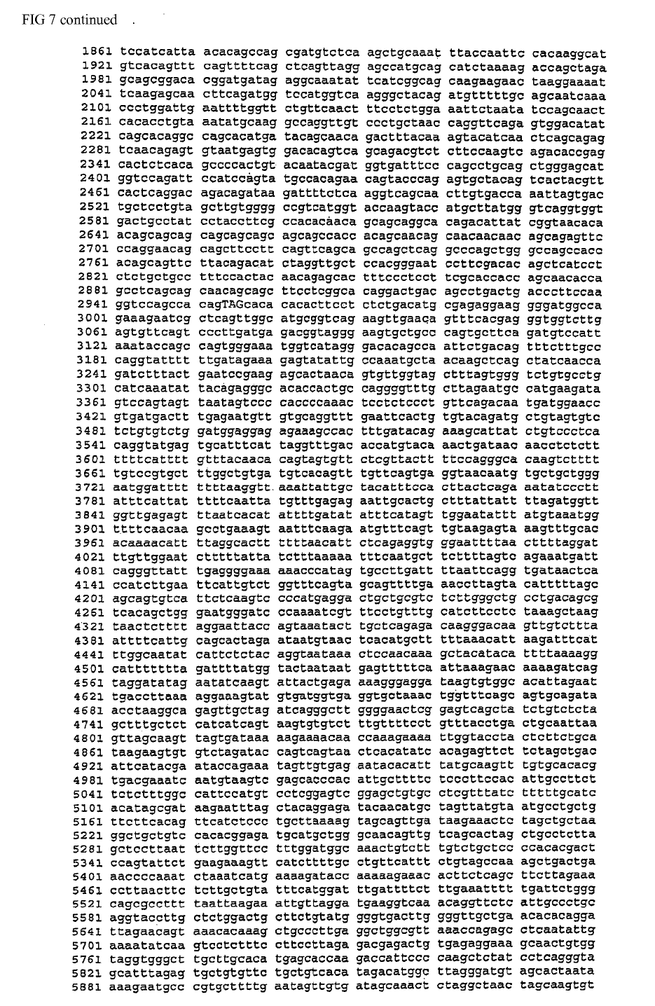

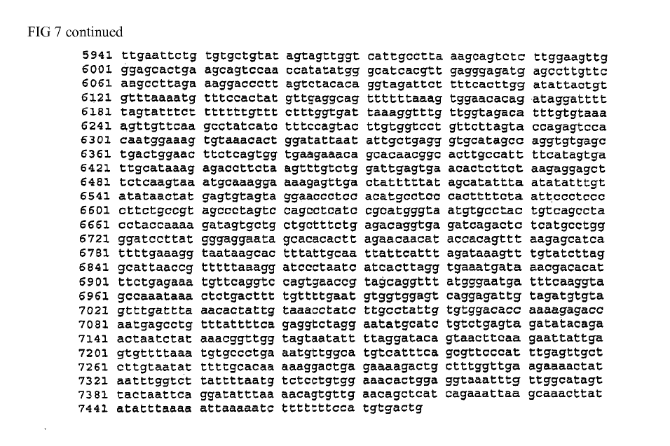

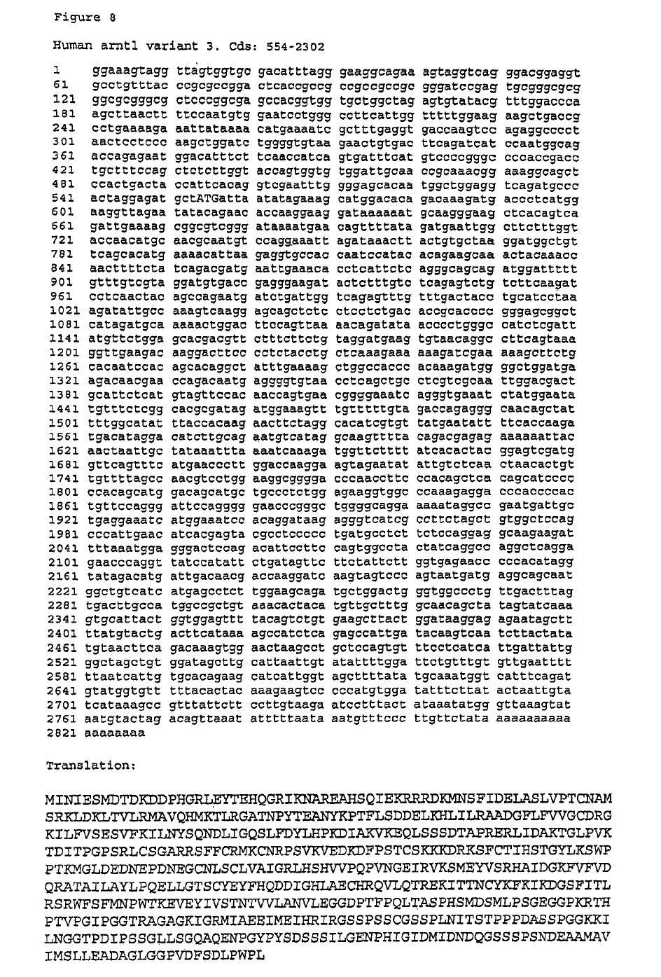

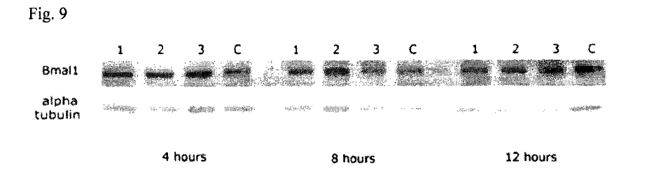

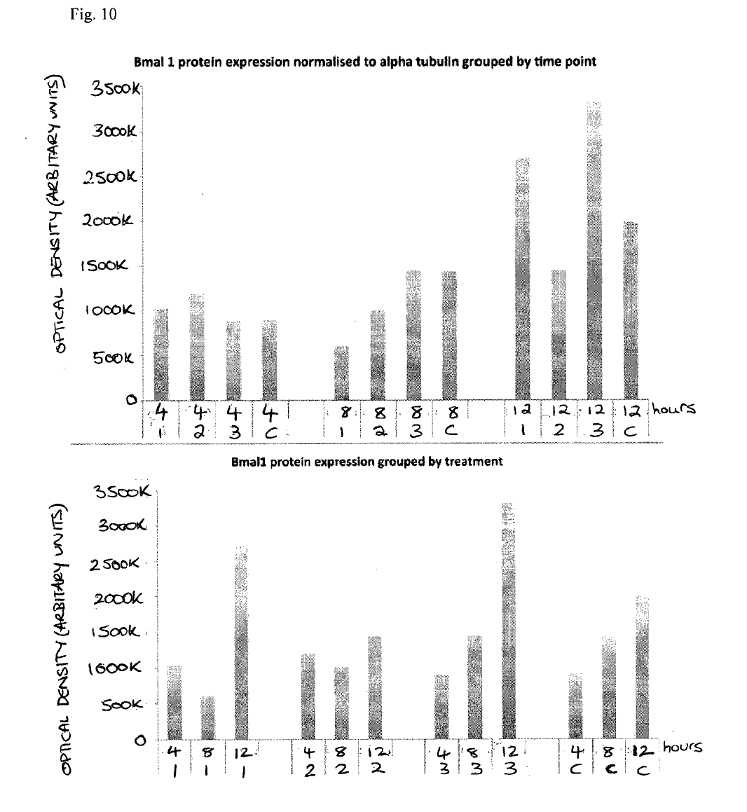

Deoxyribozymes (DNAzymes) are single stranded DNA oligos that contain an autocatalytic core sequence (5′-GGCTAGCTACAACGA-3′) flanked by 8-base arm sequences that are antisense to specific mRNA sequences. DNAzymes cleave mRNA at x-U sites, where x is any base, though the most efficient cleavage sites are AU and GU. Analysis of the BMAL1 mRNA sequence of mouse and human revealed approximately 200 putative deoxyribozyme preferred cleavage sequences (Purine-U) in the coding region, with about 300 in the CLOCK sequences. After rejection of sequences based on NIH NCBI BLAST homology to other genes, primer dimerization, secondary structure and melting temperature, about 40-50 DNAzymes for each of human and mouse BMAL1 were available to test. The results of testing the DNAzymes are shown in FIGS. 1 and 2.

1.-20. (canceled)

21. A method of treating a wound comprising administering to a subject having a wound a therapeutically effective amount of a composition comprising one or more of a polynucleotide, a vector, or an agent that alters the expression of one or both of CLOCK or BMAL.

22. The method of claim 21, wherein the composition comprises an isolated polynucleotide comprising a nucleotide sequence having substantial homology to any of the following nucleotide sequences:
(SEQ ID NO: 1)
cattcttgatccttcc,
(SEQ ID NO: 2)
catcgttatgggacta,
(SEQ ID NO: 3)
gcatctgcttccaaca,
(SEQ ID NO: 4)
tggaaggaatgtctgg,
(SEQ ID NO: 5)
atgaaaatactcataa,
(SEQ ID NO: 9)
catcgttaggctagctacaacgatgggacta,
(SEQ ID NO: 7)
cttttcaatctgactg,
(SEQ ID NO: 8)
gatgaccctcttatcc,
(SEQ ID NO: 6)
cttttcaaggctagctacaacgactgactgt
(SEQ ID NO: 10)
gtgataaaagaaccat,
(SEQ ID NO: 11)
gggttcatgaaagtga,
(SEQ ID NO: 12)
tccaccaaggctagctacaacgaccatcaaa,
and
(SEQ ID NO: 13)
gtcaacaaggctagctacaacgatgagctca.

23. The method of claim 21, wherein the composition comprises a polynucleotide between 14 and 34 bases in length.

24. The method of claim 21, wherein the composition comprises an isolated polynucleotide comprising between 14 and 45 nucleotides which is antisense to CLOCK or BMAL.

25. The method of claim 21, wherein the composition comprises a polynucleotide comprising a binding region which comprises a sequence of nucleotides which hybridizes to CLOCK or BMAL mRNA, flanked by one or more flanking regions.

26. The method of claim 25, wherein the composition comprises a polynucleotide wherein the polynucleotide comprises two binding regions which hybridize to CLOCK or BMAL mRNA, the binding regions flanking a cutting region having ligating activity.

27. The method of claim 26, wherein the composition comprises a polynucleotide, wherein the cutting region comprises the following nucleotide sequence:
(SEQ ID NO: 41)
ggctagctacaacga.

28. The method of claim 21, wherein the composition comprises a polynucleotide, consisting of one of the following sequences:
(SEQ ID NO: 1)
cattcttgatccttcc,
(SEQ ID NO: 2)
catcgttatgggacta,
(SEQ ID NO: 3)
gcatctgcttccaaca,
(SEQ ID NO: 4)
tggaaggaatgtctgg,
(SEQ ID NO: 5)
atgaaaatactcataa,
(SEQ ID NO: 9)
catcgttaggctagctacaacgatgggacta,
(SEQ ID NO: 7)
cttttcaatctgactg,
(SEQ ID NO: 8)
gatgaccctcttatcc,
(SEQ ID NO: 6)
cttttcaaggctagctacaacgactgactgt
(SEQ ID NO: 10)
gtgataaaagaaccat,
(SEQ ID NO: 11)
gggttcatgaaagtga,
(SEQ ID NO: 12)
tccaccaaggctagctacaacgaccatcaaa,
and
(SEQ ID NO: 13)
gtcaacaaggctagctacaacgatgagctca.

29. The method of claim 21, wherein the composition comprises a polynucleotide according to claim 22, comprising one of the following sequences:
(SEQ ID NO: 15)
tccttccttggtgttctgcatattctaacc,
(SEQ ID NO: 14)
atccttccttggtgttctgcatattctaac,
(SEQ ID NO: 16)
ccttggtgttctgcatattctaaccttcca,
(SEQ ID NO: 17)
gatccttccttggtgttctgcatattctaa,
(SEQ ID NO: 18)
ttccttggtgttctgcatattctaaccttc,
(SEQ ID NO: 19)
tccttggtgttctgcatattctaaccttcc,
(SEQ ID NO: 20)
atctgcttccaagaggctcatgatgacagc,
(SEQ ID NO: 21)
cttggtgttctgcatattctaaccttcca,
(SEQ ID NO: 22)
ccttccttggtgttctgcatattctaacc,
(SEQ ID NO: 23)
cttccttggtgttctgcatattctaacc,
(SEQ ID NO: 24)
gagtccctccatttagaatcttcttgcc,
(SEQ ID NO: 25)
gcttccaagaggctcatgatgacagcca,
(SEQ ID NO: 26)
ttccttggtgttctgcatattctaacc,
(SEQ ID NO: 27)
tctgtaaaacttgcctgtgacattc,
(SEQ ID NO: 28)
gtctgtaaaacttgcctgtgacattc,
(SEQ ID NO: 29)
tccttggtgttctgcatattctaacc,
(SEQ ID NO: 30)
gttactgggactacttgatccttgg,
and
(SEQ ID NO: 31)
gagtccctccatttagaatcttcttg.

30. The method of claim 21, wherein the composition comprises a vector and a pharmaceutically acceptable carrier or excipient.

31. The method of claim 21, wherein the composition comprises a pharmaceutical composition comprising a polynucleotide and a pharmaceutically acceptable carrier or excipient.

32. The method of claim 21, wherein the composition comprises a polynucleotide formulated for use in the treatment of wounds.

33. The method of claim 21, wherein the composition comprises a vector comprising a promoter or repressor of CLOCK or BMAL for use in the modulation of wound healing.

34. The method of claim 21, wherein the composition comprises a polynucleotide which is antisense to CLOCK or BMAL mRNA formulated use in the treatment of wounds.

35. The method of claim 21, wherein the composition comprises an agent which alters, increases or reduces the expression of CLOCK or BMAL for use in treating wounds.

36. The method of claim 21, wherein the method further comprises administering a wound dressing comprising a polynucleotide.


SEQUENCE LISTING (41 sequences)

SEQ ID NO: 1 - DNA, 16 bp, Mus musculus
cattcttgatccttcc

SEQ ID NO: 2 - DNA, 16 bp, Mus musculus
catcgttatgggacta

SEQ ID NO: 3 - DNA, 16 bp, Mus musculus
gcatctgcttccaaca

SEQ ID NO: 4 - DNA, 16 bp, Mus musculus
tggaaggaatgtctgg

SEQ ID NO: 5 - DNA, 16 bp, Mus musculus
atgaaaatactcataa

SEQ ID NO: 6 - DNA, 31 bp, Mus musculus
cttttcaaggctagctacaacgactgactgt

SEQ ID NO: 7 - DNA, 16 bp, Mus musculus
cttttcaatctgactg

SEQ ID NO: 8 - DNA, 16 bp, Mus musculus
gatgaccctcttatcc

SEQ ID NO: 9 - DNA, 31 bp, Mus musculus
catcgttaggctagctacaacgatgggacta

SEQ ID NO: 10 - DNA, 16 bp, Mus musculus
gtgataaaagaaccat

SEQ ID NO: 11 - DNA, 16 bp, Mus musculus
gggttcatgaaagtga

SEQ ID NO: 12 - DNA, 31 bp, Mus musculus
tccaccaaggctagctacaacgaccatcaaa

SEQ ID NO: 13 - DNA, 31 bp, Mus musculus
gtcaacaaggctagctacaacgatgagctca

SEQ ID NO: 14 - DNA, 30 bp, Mus musculus
atccttccttggtgttctgcatattctaac

SEQ ID NO: 15 - DNA, 30 bp, Mus musculus
tccttccttggtgttctgcatattctaacc

SEQ ID NO: 16 - DNA, 30 bp, Mus musculus
ccttggtgttctgcatattctaaccttcca

SEQ ID NO: 17 - DNA, 30 bp, Mus musculus
gatccttccttggtgttctgcatattctaa

SEQ ID NO: 18 - DNA, 30 bp, Mus musculus
ttccttggtgttctgcatattctaaccttc

SEQ ID NO: 19 - DNA, 30 bp, Mus musculus
tccttggtgttctgcatattctaaccttcc

SEQ ID NO: 20 - DNA, 30 bp, Mus musculus
atctgcttccaagaggctcatgatgacagc

SEQ ID NO: 21 - DNA, 29 bp, Mus musculus
cttggtgttctgcatattctaaccttcca

SEQ ID NO: 22 - DNA, 29 bp, Mus musculus
ccttccttggtgttctgcatattctaacc

SEQ ID NO: 23 - DNA, 28 bp, Mus musculus
cttccttggtgttctgcatattctaacc

SEQ ID NO: 24 - DNA, 28 bp, Mus musculus
gagtccctccatttagaatcttcttgcc

SEQ ID NO: 25 - DNA, 28 bp, Mus musculus
gcttccaagaggctcatgatgacagcca

SEQ ID NO: 26 - DNA, 27 bp, Mus musculus
ttccttggtgttctgcatattctaacc

SEQ ID NO: 27 - DNA, 25 bp, Mus musculus
tctgtaaaacttgcctgtgacattc

SEQ ID NO: 28 - DNA, 26 bp, Mus musculus
gtctgtaaaacttgcctgtgacattc

SEQ ID NO: 29 - DNA, 26 bp, Mus musculus
tccttggtgttctgcatattctaacc

SEQ ID NO: 30 - DNA, 25 bp, Mus musculus
gttactgggactacttgatccttgg

SEQ ID NO: 31 - DNA, 26 bp, Mus musculus
gagtccctccatttagaatcttcttg

SEQ ID NO: 32 - DNA, 2904 bp, Mus musculus
ggccgggcctgggccggcggggagcggattggtcggaaagtaggttagtggtgcgacatttagggaaggcagaaagtaggtcagggacggaggtgcctgtttacccgcgccggacttgccgccgccgccgccgcgggatccgagtgcgggtgcgggcgcgggcgctcgcagcgagccacggtgttgttggctgcagtgaatgcttttggaaccaaacttaaggcttcaaatgtggaaccctaggccttcattggattttggaagaagttgactgcctggaaggaagttacaaaacatgaaagtcactttggggtgaccagcaactacagtggccctttgcatctcccacactggatcgaagcttaagaactgtaacttcagatgtgggaatttgggcccagagggtgggagtgacagccccagtcaaatcctgtggacctttgagccctttgaggcccactgggcaacccctcttcaccagggtttgaagttagagtcccttgcaagcaccttccttccaatggcggaccagagaatggacatttcctcaaccatcagcgacttcatgtctccgggccccaccgacctactctccggttccctgggcaccagtggtgtggactgcaatcgcaagaggaaaggcagtgccactgactaccaagaaagtatggacacagacaaagatgaccctcatggaaggttagaatatgcagaacaccaaggaaggatcaagaatgcaagggaggcccacagtcagattgaaaagaggcgtcgggacaaaatgaacagtttcattgatgaattggcttctttggtaccaacatgcaatgcaatgtccaggaagttagataaactcaccgtgctaaggatggctgttcagcacatgaaaactttgagaggtgccaccaacccatacacagaagcaaactacaagccaacatttctatcagatgacgaactgaaacacctaattctcagggcagcagatggatttttgtttgtcgtaggatgtgaccgagggaagatcctctttgtctccgagtctgtcttcaagatcctcaattatagccagaatgaccttattggccagagcttgtttgactacctgcatccaaaagatattgccaaagttaaggaacagctatcttcctcggacactgcgccccgggagcgactcattgatgccaagactggacttccggttaaaacggatataacccctgggccctcccggctatgctctggagcccgccgctctttcttctgtagaatgaagtgcaacaggccttcagtaaaggtggaagataaggacttcgcctctacctgttcaaagaaaaaagcagatcgaaaaagcttctgcacaatccacagcacaggctatttgaaaagctggccacccacgaagatggggctggacgaagacaatgagccagacaacgagggctgcaacctcagctgcctcgttgcaatcgggcgcctgcactcgcacatggttccacaaccagcgaacggggaaatacgggtgaaatctatggagtacgtttctcgacacgcaatagatgggaaatttgtttttgtagatcagagggcgacagctattttggcgtatctaccacaggaacttctaggtacatcatgttatgagtattttcatcaagacgacataggacacctcgcagaatgtcacaggcaagttttacagacaagagaaaagatcacaactaattgctataagtttaagatcaaagatggttcttttatcacgctacgaagtcgatggttcagtttcatgaacccgtggaccaaggaagttgaatacattgtctcaaccaacactgttgttttagccaatgtcctggaaggcggggacccaaccttcccgcagctaacagcacccccccacagcatggacagcatgctgccctctggagaaggtggcccaaagaggactcatcccactgtcccaggcattccagggggaaccagagccggagcaggaaaaataggtcgaatgatcgcggaggaaatcatggaaatccacaggataagagggtcatcgccttccagctgtggctccagcccgctgaacatcacaagtacgcctccccctgatgcctcttctccaggaggcaagaagattctaaatggagggactccagacattccttccactggactattaccagggcaggctcaggagaccccagggtatccctattctgatagttcttctattcttggtgagaacccccacataggcatcgatatgatagataacgaccaaggatcaagtagtcccagtaacgatgaggcagcaatggctgtcatcatgagcctcttggaagcagatgcggggctgggtggccccgttgactttagtgacttgccatggccgctgtagacactacatttgctttggcaacagctgcagtatcaaagtgcattaatggtgaagttttacagtctgtgaagcttactggatagagagagaacagcttttatgtactgactccataaaagccacctcagagccattgatacaagtcaatctaccatgtgtaacttcagacaaagtggaactaaacctgctccagtgtttcctcatcattgagtttgggctagctgtggatagcttgcattaattgtatattttggattttgtttgtgttgaattttttaatcattgtgcacagaagcatcattggtagcttttatatgcaaaatggtcatttcagatgtatggtgtttttacactacaaagaagtcccccatgtggatatttcttatactaattgtatcataaagctgtttattcttccttgtaagaatcctttactataaatatgggttaaagtataatgtattagacagttaaatatttttaataaatgtttcccttgttct

SEQ ID NO: 33 - protein, 626 aa, Mus musculus
MADQRMDISSTISDFMSPGPTDLLSGSLGTSGVDCNRKRKGSATDYQESMDTDKDDPHGRLEYAEHQGRIKNAREAHSQIEKRRRDKMNSFIDELASLVPTCNAMSRKLDKLTVLRMAVQHMKTLRGATNPYTEANYKPTFLSDDELKHLILRAADGFLFVVGCDRGKILFVSESVFKILNYSQNDLIGQSLFDYLHPKDIAKVKEQLSSSDTAPRERLIDAKTGLPVKTDITPGPSRLCSGARRSFFCRMKCNRPSVKVEDKDFASTCSKKKADRKSFCTIHSTGYLKSWPPTKMGLDEDNEPDNEGCNLSCLVAIGRLHSHMVPQPANGEIRVKSMEYVSRHAIDGKFVFVDQRATAILAYLPQELLGTSCYEYFHQDDIGHLAECHRQVLQTREKITTNCYKFKIKDGSFITLRSRWFSFMNPWTKEVEYIVSTNTVVLANVLEGGDPTFPQLTAPPHSMDSMLPSGEGGPKRTHPTVPGIPGGTRAGAGKIGRMIAEEIMEIHRIRGSSPSSCGSSPLNITSTPPPDASSPGGKKILNGGTPDIPSTGLLPGQAQETPGYPYSDSSSILGENPHIGIDMIDNDQGSSSPSNDEAAMAVIMSLLEADAGLGGPVDFSDLPWPL

SEQ ID NO: 34 - DNA, 5801 bp, Homo sapiens
gagcgagagcgcgaaggaaatctggccgccgccgccgcgagcgctcccgaatttttacttgttcctgcaaagctgctggagctcagaagctgattctatcacattgtaagatgcctttggataattctacagtcctcttaaatgaatctttagaacttggcaagtctcactagataccttcaatcatcattttgagctcaaagaattctgagacttatggttggtcatatagaagagtaccttgaacctatagtttcctgaagaatcagtttaaaagatccaaggagtacaaaaggagaagtacaaatgtctactacaagacgaaaacgtagtatgttatgttgtttaccgtaagctgtagtaaaatgagctcgattgttgacagagatgacagtagtatttttgatgggttggtggaagaagatgacaaggacaaagcgaaaagagtatctagaaacaaatctgaaaagaaacgtagagatcaatttaatgttctcattaaagaactgggatccatgcttcctggtaatgctagaaagatggacaaatctactgttctgcagaaaagcattgattttttacgaaaacataaagaaatcactgcacagtcagatgctagtgaaattcgacaggactggaaacctacattccttagtaatgaagagtttacacaattaatgttagaggctcttgatggtttttttttagcaatcatgacagatggaagcataatatatgtgtctgagagtgtaacttcattacttgaacatttaccatctgatcttgtggatcaaagtatatttaattttatcccagaaggggaacattcagaggtttataaaatactctctactcatctgctggaaagtgattcattaaccccagaatatttaaaatcaaaaaatcagttagaattctgttgtcacatgctgcgaggaacaatagacccaaaggagccatctacctatgaatatgtaaaatttataggaaatttcaaatctttaaacagtgtatcctcttcagcacacaatggttttgaaggaactatacaacgcacacataggccatcttatgaagatagagtttgttttgtagctactgtcaggttagctacacctcagttcatcaaggaaatgtgcactgttgaagaacccaatgaagagtttacatctagacatagtttagaatggaagtttctgtttctagatcacagggcaccacccataatagggtatttgccatttgaagttctgggaacatcaggctatgattactatcatgtggatgacctagaaaatttggcaaaatgtcatgagcacttaatgcaatatgggaaaggcaaatcatgttattataggttcctgactaaggggcaacagtggatttggcttcagactcattattatatcacttaccatcagtggaattcaaggccagagtttattgtttgtactcacactgtagtaagttatgcagaagttagggctgaaagacgacgagaacttggcattgaagagtctcttcctgagacagctgctgacaaaagccaagattctgggtcagataatcgtataaacacagtcagtctcaaggaagcattggaaaggtttgatcacagcccaaccccttctgcctcttctcggagttcaagaaaatcatctcacacggccgtctcagacccttcctcaacaccaaccaagatcccgacggatacgagcactccacccaggcagcatttaccagctcatgagaagatggtgcaaagaaggtcatcatttagtagtcagtccataaattcccagtctgttggttcatcattaacacagccagtgatgtctcaagctacaaatttaccaattccacaaggcatgtcccagtttcagttttcagctcaattaggagccatgcaacatctgaaagaccaattggaacaacggacacgcatgatagaagcaaatattcatcggcaacaagaagaactaagaaaaattcaagaacaacttcagatggtccatggtcaggggctgcagatgtttttgcaacaatcaaatcctgggttgaattttggttccgttcaactttcttctggaaattcatctaacatccagcaacttgcacctataaatatgcaaggccaagttgttcctactaaccagattcaaagtggaatgaatactggacacattggcacaactcagcacatgatacaacaacagactttacagagtacatcaactcagagtcaacaaaatgtactgagtgggcacagtcagcaaacatctctacccagtcagacacagagcactcttacagccccactgtataacactatggtgatttctcagcctgcagccggaagcatggtccagattccatctagtatgccacaaaacagcacccagagtgctgcagtaactacattcactcaggacaggcagataagattttctcaaggtcaacaacttgtgaccaaattagtgactgctcctgtagcttgtggggcagtcatggtacctagtactatgcttatgggccaggtggtgactgcatatcctacttttgctacacaacagcaacagtcacagacattgtcagtaacgcagcagcagcagcagcagagctcccaggagcagcagctcacttcagttcagcaaccatctcaggctcagctgacccagccaccgcaacaatttttacagacttctaggttgctccatgggaatccctcaactcaactcattctctctgctgcatttcctctacaacagagcaccttccctcagtcacatcaccagcaacatcagtctcagcaacagcagcaactcagccggcacaggactgacagcttgcccgacccttccaaggttcaaccacagtagcacacgtgcttcctctcttgacatcaagggaggaaggggatggcccattaagagttactcagatgacctgaggaaaggagggaaagttccagcagtttcatgagatgcagtattgagtgttctagttcctggaattagttggcagagaaaatgctgcctagtgctacagatgtacattaaataccagccagcaggaggtgatcataggggcatagccagttctgacagtgttttaggtgcctggatattttttgatggaaaaagaatatattgccaaatattaagaagctcagctatgaaatgacctccagggaatcagaaaggcactaatgatgttagtaacttttagtggttctgtgcctcttatcaagtgttacagaggacataccactgccatgtcaggggtttgcttacagtgatgccatgaagacagtccagtagacttggtagcgaccccctcccccaacccctctcccttttcagataatgatggaacagtaattactttcagaatgttgtgtgggttcaaattctctatgtacagatgatgtaaaaatatgtatatgtctagataaaaggagagaaagcaaaacattttgtatgctgcatgaaagcgttatctcttccttacaggtgtgagcacctttcctgaaattctgacaccatgtgcaaactgatccatcctgtttttccttttgtttacaacacagtagtgttctgttcacttttccggggcacaagtttttttgttcatactttggctgtgatgtcacagtttgttcagtgaggtatgatgtgctgctgggaatggattttttttttcaggttaaattattgatacaacaggattttcaagttattcagaaatatccctcatttcattatttttcaattatgtttgaaaataggatttgcactgctttattttaggtggctgggagttttgattgcatattttgttatagttcatagttggaaatatttgcgtaaatggttttcaacaagcctgaaagtaatttcaagaatgtttcagttatagaggtaaaatttgcacacaaaacatcttaggcactttttaacattctcaatcatgggaattttaacttttgggatttgttgaaatcttttttattatccttcacaatttcaatgcttcttttagtcagaaatgattcagggttatttgaggggaaaaaaccccatagtgccttgattttaattcaggtgataactcaccatcttgaagtcattgtccggtttccgtagcagttttgaaaccttagtacctttttaacagcatgtgggtgtcagtgtcattattagtctcctaataagttcctctgaagactgctatcagtctcttggactggaggtacaaataatttagaaataaaagatgataacctaacactatcatagttattaatgtgatcctaaaattgtttcctaaatcagcatttttctttagtcatttaagaatttaccagaaatatttgctcaatatgatcttgatattcctacaaagaaaaaagaaggggtagggatttggctatgccttcactacaacattagaatattgtaactcacatgccttctaaacgtgaactaagatttcctttggcaatatcatattctaaaagtaataaattccaatacaagttacatacatttaaaaaacattttacagattttatggtactaatgaaatttacagtgatagaacaaaagaggattagtagaaaatacattattagaatataaaaaatgttattactgaggaaagggaggagaggacaagtgtaataaatcaaaattgacctcaaaagaaaatgtgtaacagagttgaggttgttaaaacagaaaaggttctgaataatgaagattaacctaatgcagaattgctaggtaaagaggtcaggggaatgctaagccagttcttaagacttctctgtcctctgctttgctgttatccttaaggcatatactttgtctttctgcagaaaattctacctggctacaattactttgaacattaatgttgaaaaagaaaacaaccaaagaaaattggtacttacccttctacaaaagaagtgtgactagatatcaatcagtaattaacatatcaaggagctcttctagctaaatgaccatccagtagagatttcccacattcccatgaatatcaagaatagttgtcagaatatgtatgtacctgagcatatgtacacagacaagggggatgttgtggaatatggcaatagcattgttcttctcccctttcaaattgcctttcttgaccttatgccattccatatatatctgagttgtgcctcatttatttattggcaatacctagtgatacggatttagctaacaaaagatatgaagaactattatattgaggcctgtcctctacataccacacttaaaagatggtgaactgtgagtactacttaggttgacagcaacaaagcataagacaagccccaggtaaacgtctaaactgtttactcacattgtcctactccagccccttcaattatttcccatctccacaaatagtcgggggaaaaaattaaaattttcctttatgattcttactgttcttcgcagctcatcttttcctgcttagaattaaccattgctaatttaaaggagcagctagctgcttttctgtcagtctgaagcgtagtagtggaagaggtagtaagcaccagctgcctctttgctgctttgttttcctcctgattctcttaaatttgggttgcaaagctatcccgccccccaccctgccccatgaaacttgagcattcaaatgaagattcagcagtgtctgttcttcatttctatagccaaagctgttagttaaaatcccaaatctatagcatttaaagataccaaatagaaacaccttccagcttt

SEQ ID NO: 35 - DNA, 7478 bp, Mus musculus
ggggaggagcgcggcggtagcggtgaattttgaggggtgggtcgggggcgcgcactcgccgcccctggtgctgccggctcccggagccgtggcgtgtcccggctgtcgccgctcggctgtcgcgagccgccgcgggcagagtcccgggcgggggagggaggaagccggagcctcaggcacgtgaaagaaaagcacaagaagaaacttttacaggcgttgttgattggactagggcaacgattcccaaaatcaccagcaagagttctgatggtcagtcacacagaagacggccttgcgtctgtgggtgttggagactccattctaaagatataaaaagtgaaagaggagaagtacaaatgtctaccacaagacgaaaacataatgtgttatggtgtttaccgtaagctgtagtaaaatgagctcaattgttgacagagatgacagtagtatttttgatggattggtggaagaagatgacaaggacaaagcaaaaagagtatctagaaacaaatcagaaaagaaacgtagagatcagttcaatgtcctcattaaggagctggggtctatgcttcctggtaacgcgagaaagatggacaagtctactgttctacagaagagcattgattttttgcgcaaacataaagagaccactgcacagtcagatgctagtgagattcgacaggactggaaacccacattccttagtaatgaagagtttacacagttaatgttagaggctcttgatggtttttttttagcgatcatgacagatggaagtataatatatgtatctgagagtgtaacttcgttacttgaacatttaccatctgatcttgtggatcaaagtatatttaattttatcccagagggagaacattcagaggtttataagatactctctactcatctgctggaaagtgactcattaacccctgagtacttaaaatcaaaaaatcagttagaattctgttgtcacatgcttcgaggaacaatagacccaaaggagccatccacctatgaatatgtgagatttataggaaattttaaatctttaaccagtgtatcaacttcaacacacaatggttttgaaggaactatacaacgcacacataggccttcttatgaagatagagtttgttttgtagctactgtcagattagctacacctcagttcatcaaggaaatgtgtactgttgaagaaccaaatgaagagtttacatctagacacagtttagaatggaagtttctatttttagatcacagggcaccaccaataataggctatttgccatttgaagtcttgggaacatcaggctatgattactatcatgtggatgacctagaaaatctggcaaaatgtcacgagcacttaatgcaatatggaaaaggcaaatcgtgttactatagattcctgaccaaaggccagcagtggatatggcttcagactcattattatattacttaccatcagtggaattcaaggccagagttcattgtttgtactcacactgtagtaagttatgcagaagttagggctgaaagacggcgagaacttggcattgaagagtctcttcctgagacagctgctgacaaaagccaagattctgggtctgacaatcgtatcaacacagtgagtctcaaggaagcactggaaaggtttgatcacagcccaactccttctgcctcctctagaagctcacgaaagtcatctcacaccgcagtctcagacccttcctccacaccgacaaagatccctactgatactagcactcctcccagacagcatttgccagctcatgaaaagatgacacagcggaggtcgtccttcagcagtcagtccataaactcccagtcagttggtccatcattaacacagccagcgatgtctcaagctgcaaatttaccaattccacaaggcatgtcacagtttcagttttcagctcagttaggagccatgcagcatctaaaagaccagctagagcagcggacacggatgatagaggcaaatattcatcggcagcaagaagaactaaggaaaattcaagagcaacttcagatggtccatggtcaagggctacagatgtttttgcagcaatcaaaccctggattgaattttggttctgttcaactttcctctggaaattctaatatccagcaactcacacctgtaaatatgcaaggccaggttgtccctgctaaccaggttcagagtggacatatcagcacaggccagcacatgatacagcaacagactttacaaagtacatcaactcagcagagtcaacagagtgtaatgagtggacacagtcagcagacgtctcttccaagtcagacaccgagcactctcacagccccactgtacaatacgatggtgatttcccagcctgcagctgggagcatggtccagattccatccagtatgccacagaacagtacccagagtgctacagtcactacgttcactcaggacagacagataagattttctcaaggtcagcaacttgtgaccaaattagtgactgctcctgtagcttgtggggccgtcatggtaccaagtaccatgcttatgggtcaggtggtgactgcctatcctaccttcgccacacaacagcagcaggcacagacattatcggtaacacaacagcagcagcagcagcagcagcagccaccacagcaacagcaacaacaacagcagagttcccaggaacagcagcttccttcagttcagcagccagctcaggcccagctgggccagccaccacagcagttcttacagacatctaggttgctccacgggaatccttcgacacagctcatcctctctgctgcctttccactacaacagagcactttccctccttcgcaccaccagcaacaccagcctcagcagcaacagcagcttcctcggcacaggactgacagcctgactgacccttccaaggtccagccacagtagcacacacacttcctctctgacatgcgagaggaaggggatggccagaaagaatcgctcagttggcatgcggtcagaagttgaacagtttcacgagggtggtcttgagtgttcagtcccttgatgagacggtagggaagtgctgcccagtgcttcagatgtccattaaataccagccagtgggaaatggtcatagggacacagccaattctgacagtttctttgcccaggtattttttgatagaaagagtatattgccaaatgctaacaagctcagctatcaaccagatctttactgaatccgaagagcactaacagtgttggtagctttagtgggtctgtgcctgcatcaaatattacagagggcacaccactgccaggggtttgcttagaatgccatgaagatagtccagtagttaatagtccccaccccaaactcctctccctgttcagacaatgatggaaccgtgatgactttgagaatgttgtgcaggtttgaattcactgtgtacagatgctgtagtgtctctgtgtctggatggaggagagaaagccactttgatacagaaagcattatctgtccctcacaggtatgagtgcatttcattaggtttgacaccatgtacaaactgataacaacctctcttttttcattttgtttacaacacagtagtgttctcgttacttttccagggcacaagtctttttgtccgtgctttggctgtgatgtcacagtttgttcagtgaggtaacaatgtgctgctgggaatggattttttttaaggttaaattattgctacatttccacttactcagaaatatcccttatttcattatttttcaattatgtttgagagaattgcactgctttattattttagatggttggttgagagtttaatcacatattttgatatatttcatagttggaatatttatgtaaatggttttcaacaagcctgaaagtaatttcaagaatgtttcagttgtaagagtaaagtttgcacacaaaacattttaggcacttttttaacattctcagaggtgggaattttaacttttaggatttgttggaatctttttattatctttaaaaatttcaatgcttcttttagtcagaaatgattcagggttatttgaggggaaaaaacccatagtgccttgattttaattcaggtgataactcaccatcttgaattcattgtctggtttcagtagcagttttgaaaccttagtacatttttagcagcagtgtcattctcaagtccccatgaggactgctgcgtctcttgggctgcctgacagcgtcacagctgggaatgggatcccaaaatcgtttcctgtttgcatcttcctctaaagctaagtaactcttttaggaattaccagtaaatacttgctcagagacaagggacaagttgtctttaattttcattgcagcactagaataatgtaactcacatgctttttaaacattaagatttcatttggcaatatcattctctacaggtaataaactccaacaaagctacatacattttaaaaggcatttttttagattttatggtactaataatgagtttttcaattaaagaacaaaagatcagtaggatatagaatatcaagtattactgagaaaagggaggataagtgtggcacattagaattgaccttaaaaggaaagtatgtgatggtgaggtgctaaactggtttcagcagtgcagataacctaaggcagagttgctagatcagggcttggggaactcggagtcagctatctgtctctagctttgctctcatcatcagtaagtgtgtctttgttttcctgtttacctgactgcaattaagttagcaagttagtgataaaaagaaaacaaccaaagaaaattggtacctactcttctgcataagaagtgtgtctagataccagtcagtaactcacatatcacagagttcttctagctgacattcatacgaataccagaaatagttgtgagaatacacatttatgcaagtttgtgcacacgtgacgaaatcaatgtaagtcgagcacccacattgcttttctcccttccacattgccttcttctctttggccattccatgtcctcggagtcggagctgtgcctcgtttatctttttgcatcacatagcgataagaatttagctacaggagatacaacatgctagttatgtaatgcctgctgttcttcacagttcatctccctgcttaaaagtagcagttgataagaaactctagctgctaaggctgctgtccacacggagatgcatgctgggcaacagttgtcagcactagctgcctcttagctccttaattcttggttcctttggatggcaaactgtctttgtctgctccccacacgactccagtattctgaagaaagttcatcttttgcctgttcatttctgtagccaaagctgactgaaaccccaaatctaaatcatgaaaagataccaaaaagaaacacttctcagcttcttagaaaccttaacttctcttgctgtatttcatggatttgattttctttgaaatttttgattctgggcagcgccttttaattaagaaattgttaggatgaaggtcaaacaggttctcattgccctgcaggtaccttgctctggactgcttctgtatggggtgacttggggttgctgaacacacaggattagaacagtaaacacaaagctgcccttgaggctggcgttaaaccagagcctcaatattgaaaatatcaagtcctctttccttccttagagacgagactgtgagaggaaagcaactgtggtaggtgggcttgcttgcacatgagcaccaagaccattccccaagctctatcctcagggtagcatttagagtgctgtgttctgctgtcacatagacatggcttagggatgtagcactaataaaagaatgcccgtgcttttgaatagttgtgatagcaaactctaggctaactagcaagtgtttgaattctgtgtgctgtatagtagttggtcattgccttaaagcagtctcttggaagttgggagcactgaagcagtccaaccatatatgggcatcacgttgagggagatgagccttgttcaagccttagaaaggacccttagtctacacaggtagattcttttcacttggatattactgtgtttaaaatgtttccactatgttgaggcagttttttaaagtggaacacagataggatttttagtatttctttttttgtttctttggtgattaaaggtttgttggtagacatttgtgtaaaagttgttcaagcctatcatctttccagtacttgtggtcctgttcttagtaccagagtccacaatggaaagtgtaaacactggatattaatattgctgagggtgcatagccaggtgtgagctgactggaacttctcagtggtgaagaaacagcacaacggcacttgccattttcatagtgattgcataaagagaccttctaagtttgtctggattgagtgaacactcttctaagaggagcttctcaagtaaatgcaaaggaaaagagttgactatttttatagcatatttaatatatttgtatataactatgagtgtagtaggaaccctccacatgcctcccacttttctaattccctccccttctgccgtagccctagtccagcctcatccgcatgggtaatgtgcctactgtcagcctacctaccaaaagatagtgctgctgctttctgagacaggtgagatcagactctcatgcctggggatccttatgggaggaatagcacacacttagaacaacataccacagtttaagagcatcattttgaaaggtaataagcactttattgcaattattcatttagataaagtttgtatcttaggcattaaccgtttttaaaggatccctaatcatcacttaggtgaaatgataaacgacacatttctgagaaatgttcaggtccagtgaaccgtagcaggtttatgggaatgatttcaaggtagccaaataaactctgacttttgttttgaatgtggtggagtcaggagattgtagatgtgtagtttgatttaaacactattgtaaacctatcttgcctattgtgtggacaccaaaagagaccaatgagcctgtttattttcagaggtctaggaatatgcatctgtctgagtagatatacagaactaatctataaacggttggtagtaatattttaggatacagtaacttcaagaattattgagtgttttaaatgtgccctgaaatgttggcatgtcatttcagcgttcccatttgagttgctcttgtaatatttttgcacaaaaaggactgagaaaagactgctttggttgaagaaaactataatttggtcttattttaatgtctcctgtggaaacactggaggtaaatttgttggcatagttactaattcaggatatttaaaacagtgttgaacagctcatcagaaattaagcaaacttatatatttaaaaattaaaaatctttttttccatgtgactg

SEQ ID NO: 36 - DNA, 2828 bp, Homo sapiens
ggaaagtaggttagtggtgcgacatttagggaaggcagaaagtaggtcagggacggaggtgcctgtttacccgcgccggactcaccgccgccgccgccgcgggatccgagtgcgggcgcgggcgcgggcgctcccggcgagccacggtggtgctggctagagtgtatacgtttggacccaagcttaacttttccaatgtggaatcctgggccttcattggtttttggaagaagctgaccgcctgaaaagaaattataaaacatgaaaatcgctttgaggtgaccaagtccagaggcccctaactcctcccaagctggatctggggtgtaagaactgtgacttcagatcatccaatggcagaccagagaatggacatttcttcaaccatcagtgatttcatgtccccgggccccaccgacctgctttccagctctcttggtaccagtggtgtggattgcaaccgcaaacggaaaggcagctccactgactaccattcacaggtcgaatttggggagcacaatggctggaggtcagatgcccactaggagatgctatgattaatatagaaagcatggacacagacaaagatgaccctcatggaaggttagaatatacagaacaccaaggaaggataaaaaatgcaagggaagctcacagtcagattgaaaagcggcgtcgggataaaatgaacagttttatagatgaattggcttctttggtaccaacatgcaacgcaatgtccaggaaattagataaacttactgtgctaaggatggctgttcagcacatgaaaacattaagaggtgccaccaatccatacacagaagcaaactacaaaccaacttttctatcagacgatgaattgaaacacctcattctcagggcagcagatggatttttgtttgtcgtaggatgtgaccgagggaagatactctttgtctcagagtctgtcttcaagatcctcaactacagccagaatgatctgattggtcagagtttgtttgactacctgcatcctaaagatattgccaaagtcaaggagcagctctcctcctctgacaccgcaccccgggagcggctcatagatgcaaaaactggacttccagttaaaacagatataacccctgggccatctcgattatgttctggagcacgacgttctttcttctgtaggatgaagtgtaacaggccttcagtaaaggttgaagacaaggacttcccctctacctgctcaaagaaaaaagatcgaaaaagcttctgcacaatccacagcacaggctatttgaaaagctggccacccacaaagatggggctggatgaagacaacgaaccagacaatgaggggtgtaacctcagctgcctcgtcgcaattggacgactgcattctcatgtagttccacaaccagtgaacggggaaatcagggtgaaatctatggaatatgtttctcggcacgcgatagatggaaagtttgtttttgtagaccagagggcaacagctattttggcatatttaccacaagaacttctaggcacatcgtgttatgaatattttcaccaagatgacataggacatcttgcagaatgtcataggcaagttttacagacgagagaaaaaattacaactaattgctataaatttaaaatcaaagatggttcttttatcacactacggagtcgatggttcagtttcatgaacccttggaccaaggaagtagaatatattgtctcaactaacactgttgttttagccaacgtcctggaaggcggggacccaaccttcccacagctcacagcatccccccacagcatggacagcatgctgccctctggagaaggtggcccaaagaggacccaccccactgttccagggattccagggggaacccgggctggggcaggaaaaataggccgaatgattgctgaggaaatcatggaaatccacaggataagagggtcatcgccttctagctgtggctccagcccattgaacatcacgagtacgcctccccctgatgcctcttctccaggaggcaagaagattttaaatggagggactccagacattccttccagtggcctactatcaggccaggctcaggagaacccaggttatccatattctgatagttcttctattcttggtgagaacccccacataggtatagacatgattgacaacgaccaaggatcaagtagtcccagtaatgatgaggcagcaatggctgtcatcatgagcctcttggaagcagatgctggactgggtggccctgttgactttagtgacttgccatggccgctgtaaacactacatgttgctttggcaacagctatagtatcaaagtgcattactggtggagttttacagtctgtgaagcttactggataaggagagaatagcttttatgtactgacttcataaaagccatctcagagccattgatacaagtcaatcttactatatgtaacttcagacaaagtggaactaagcctgctccagtgtttcctcatcattgattattgggctagctgtggatagcttgcattaattgtatattttggattctgtttgtgttgaattttttaatcattgtgcacagaagcatcattggtagcttttatatgcaaatggtcatttcagatgtatggtgtttttacactacaaagaagtcccccatgtggatatttcttatactaattgtatcataaagccgtttattcttccttgtaagaatcctttactataaatatgggttaaagtataatgtactagacagttaaatatttttaataaatgtttcccttgttctataaaaaaaaaaaaaaaaaaa

SEQ ID NO: 37 - protein, 582 aa, Homo sapiens
MINIESMDTDKDDPHGRLEYTEHQGRIKNAREAHSQIEKRRRDKMNSFIDELASLVPTCNAMSRKLDKLTVLRMAVQHMKTLRGATNPYTEANYKPTFLSDDELKHLILRAADGFLFVVGCDRGKILFVSESVFKILNYSQNDLIGQSLFDYLHPKDIAKVKEQLSSSDTAPRERLIDAKTGLPVKTDITPGPSRLCSGARRSFFCRMKCNRPSVKVEDKDFPSTCSKKKDRKSFCTIHSTGYLKSWPPTKMGLDEDNEPDNEGCNLSCLVAIGRLHSHVVPQPVNGEIRVKSMEYVSRHAIDGKFVFVDQRATAILAYLPQELLGTSCYEYFHQDDIGHLAECHRQVLQTREKITTNCYKFKIKDGSFITLRSRWFSFMNPWTKEVEYIVSTNTVVLANVLEGGDPTFPQLTASPHSMDSMLPSGEGGPKRTHPTVPGIPGGTRAGAGKIGRMIAEEIMEIHRIRGSSPSSCGSSPLNITSTPPPDASSPGGKKILNGGTPDIPSSGLLSGQAQENPGYPYSDSSSILGENPHIGIDMIDNDQGSSSPSNDEAAMAVIMSLLEADAGLGGPVDFSDLPWPL

SEQ ID NO: 38 - DNA, 16 bp, Mus musculus
tccaccaaccatcaaa

SEQ ID NO: 39 - DNA, 16 bp, Mus musculus
gtcaacaatgagctca

SEQ ID NO: 40 - DNA, 16 bp, Mus musculus
cttttcaactgactgt

SEQ ID NO: 41 - DNA, 15 bp, Artificial Sequence
  Description of Artificial Sequence Synthetic oligonucleotide
ggctagctacaacga